SEQUENCE LISTING (11 sequences)

SEQ ID NO: 1 - DNA, 20 bp, Artificial Sequence
  Synthetic DNA
gaaggacagaatgacgatga

SEQ ID NO: 2 - DNA, 20 bp, Artificial Sequence
  Synthetic DNA
gtgacgagggatgactgcgt

SEQ ID NO: 3 - DNA, 20 bp, Artificial Sequence
  Synthetic DNA
tcgtctatctcgttatgccc

SEQ ID NO: 4 - DNA, 3394 bp, Arabidopsis thaliana
  CDS (109)..(3159)
tttctcttcttcgcctcctcctcctccaagtgtagaacgacgattgttgaatgcgttattgtaattgttaagaattgaagttaaagtgttgtttttgaatctggtgaaatggtgtggtttcgtgctggttccagtgttacaaagctagctgttagaaggattttgaatcagggtgcttcgtatgcgacgaggacacggtctattccgtctcaaactcgttcctttcactcgactatatgcagaccaaaggctcagagtgctccagttcctagagctgttcctctttctaagctaactgatagtttcttagatgggacgagcagtgtctaccttgaggagttacaaagggcttgggaagctgatcctaacagtgtagatgagtcttgggataatttctttaggaactttgttggtcaggctgccacgtctcctggcatctctgggcagacaattcaggagagtatgaggctgttgttacttgttagggcttatcaggtgaatggtcacatgaaagcgaagttggatccgttaggtttggaacagcgagagatccctgaggatcttgacttggctctttatggattcactgaggctgaccttgacagagagttcttcttgggggtgtggcagatgtcaggattcatgtctgagaaccgaccagtgcagacccttcgttccatattgacaaggctcgaacaggcatactgtgggaatatcggatttgagtatatgcacattgcagatcgagataaatgtaactggttgagagaaaagattgagacaccaactccttggcggtacaacagggagcgccgtgaggtgattctcgatcggcttgcatggagtactcagttcgagaatttcttagctaccaagtggacaacagccaaaagatttggacttgagggaggagaatcattaattcctggaatgaaggagatgtttgacagagcagcagatcttggagtagagagtattgttattggaatgtctcacagaggaagattgaatgttctgggtaatgttgttcggaagccactccgtcagatatttagtgagttcagtggtggtattaggcctgtagatgaagttggctacactggaactggtgatgtcaaatatcacttgggaacctcttatgatcgacctacaagaggtgggaagaaaatccatctctctttggttgctaatccaagtcacttggaagctgcagattctgttgttgttggcaaaaccagagcaaaacagtactactccaatgatttagacaggaccaaaaatttaggtattttgattcacggagatggtagttttgctggacaaggggtagtctatgaaactctccatcttagtgctcttccaaactacaccaccggaggaaccatacatattgtggtgaacaaccaagtggctttcacgacagatccaagggcggggagatcttcccagtattgtactgatgttgcaaaggctttgagtgctcccatctttcatgttaatggggatgatgttgaggctgttgttcatgcctgcgagcttgctgctgagtggcgtcagacttttcattctgatgttgtcgttgatttggtttgctaccgtaggttcgggcataatgagatagatgaaccatctttcactcagccaaaaatgtacaaggttatcaaaaatcatccttcaacccttcagatctaccacaaaaagctcttggaatgcggtgaagtatcacaacaggatattgaccggatacaggaaaaggttaacaccatcctcaatgaagaatttgtcgctagtaaggactatctccctaagaaacgagattggctttcaaccaattgggctggatttaagtctcctgagcagatctcacgtgttagaaacactggcgtcaaaccagagatactgaagactgttggcaaggcaatttcatctcttccagaaaacttcaagccacacagggcagtgaagaaagtttatgaacaacgtgcccaaatgattgaatcaggagagggagttgactgggcccttgcagaagctcttgcttttgctaccttagttgtggaaggcaatcatgtccgattgagtggtcaggatgtcgaacgaggaacatttagtcatcgtcattctgtccttcatgaccaggaaactggagaagagtattgtcctctagatcatctcatcatgaatcaggatcctgagatgtttactgttagcaacagttctctttcagaatttggtgtccttgggttcgaattgggttactccatggaaagcccgaactcgttggtactatgggaagctcagtttggagacttcgccaatggagctcaggtgatatttgatcagttcatcagcagtggagaagccaaatggctgcgtcaaaccgggcttgttatgctacttccccatggttatgatggtcagggacctgaacattcaagtgcgaggttggaacgttaccttcagatgagtgatgataatccctatgtcataccagacatggaaccaacaatgcgaaagcaaattcaagaatgtaattggcagattgtcaatgccacaactcccgccaactatttccatgttctgcggcgacagatacacagagacttccgtaagcctctgattgtaatggcaccaaagaacttgctccgtcacaaggactgcaaatcaaatctctcagagtttgatgatgtccaaggccacccaggttttgacaagcaaggaactagatttaagcgattaatcaaggatcagaatgatcactctgatcttgaagaaggcatcagaagattggtactttgctccggaaaggtctattatgagcttgatgatgaacggaagaaggttggcgcaacagatgttgctatctgtagagttgaacagctttgtcctttcccatatgatctcattcagcgtgagctcaagagatatccaaatgcggagatcgtttggtgccaagaagaggcgatgaacatgggagcattcagctacatatctccacggctatggacagcaatgagaagcgtaaacagaggagatatggaagacattaagtatgttggtcgtggtccttctgctgcaactgccacgggtttctatactttccatgtcaaagagcaagccgggcttgtccagaaagccatcggaaaggaacccatcaattaaaaactcttcctttttaaatgacttctcagactgataagagaaaaaggagaaaactttgaaataagaactttgggtgatgcaaaagtgctaaacaacactgtcaaaatccgctgtttctttgatatttttttggtcccattgattttgagcctggctcgtgttgccagtcaaaacattaaaaaaaaatcttatattgatatacagttttgttattgctaaaaaaaaaaaaaaa

SEQ ID NO: 5 - protein, 1017 aa, Arabidopsis thaliana
MVWFRAGSSVTKLAVRRILNQGASYATRTRSIPSQTRSFHSTICRPKAQSAPVPRAVPLSKLTDSFLDGTSSVYLEELQRAWEADPNSVDESWDNFFRNFVGQAATSPGISGQTIQESMRLLLLVRAYQVNGHMKAKLDPLGLEQREIPEDLDLALYGFTEADLDREFFLGVWQMSGFMSENRPVQTLRSILTRLEQAYCGNIGFEYMHIADRDKCNWLREKIETPTPWRYNRERREVILDRLAWSTQFENFLATKWTTAKRFGLEGGESLIPGMKEMFDRAADLGVESIVIGMSHRGRLNVLGNVVRKPLRQIFSEFSGGIRPVDEVGYTGTGDVKYHLGTSYDRPTRGGKKIHLSLVANPSHLEAADSVVVGKTRAKQYYSNDLDRTKNLGILIHGDGSFAGQGVVYETLHLSALPNYTTGGTIHIVVNNQVAFTTDPRAGRSSQYCTDVAKALSAPIFHVNGDDVEAVVHACELAAEWRQTFHSDVVVDLVCYRRFGHNEIDEPSFTQPKMYKVIKNHPSTLQIYHKKLLECGEVSQQDIDRIQEKVNTILNEEFVASKDYLPKKRDWLSTNWAGFKSPEQISRVRNTGVKPEILKTVGKAISSLPENFKPHRAVKKVYEQRAQMIESGEGVDWALAEALAFATLVVEGNHVRLSGQDVERGTFSHRHSVLHDQETGEEYCPLDHLIMNQDPEMFTVSNSSLSEFGVLGFELGYSMESPNSLVLWEAQFGDFANGAQVIFDQFISSGEAKWLRQTGLVMLLPHGYDGQGPEHSSARLERYLQMSDDNPYVIPDMEPTMRKQIQECNWQIVNATTPANYFHVLRRQIHRDFRKPLIVMAPKNLLRHKDCKSNLSEFDDVQGHPGFDKQGTRFKRLIKDQNDHSDLEEGIRRLVLCSGKVYYELDDERKKVGATDVAICRVEQLCPFPYDLIQRELKRYPNAEIVWCQEEAMNMGAFSYISPRLWTAMRSVNRGDMEDIKYVGRGPSAATATGFYTFHVKEQAGLVQKAIGKEPIN

SEQ ID NO: 6 - DNA, 3412 bp, Arabidopsis thaliana
  CDS (78)..(3152)
ttcagagattaaacaatttgaaaaatcggagactctgggattgtatggttcgttgttactgatagattacttaagctatggtttggtttagaatcggttcttctgtggcaaagcttgccataagaaggacactgtctcagtctcgttgtggttcatatgccactagaacaagggttttgccttgtcaaaccagatgttttcactctacaatactcaaatcaaaggcagagtctgctgcacctgttccacgtcctgtcccactttctaagctaactgatagcttcttagatggaacaagcagtgtgtatctagaggagttacaaagagcttgggaggctgatcccaacagtgttgatgagtcgtgggataacttttttaggaattttgtgggtcaggcttctacatcgcctggtatctcggggcaaaccattcaagaaagcatgcgtttgttgttgctagttagagcttaccaggttaatggccacatgaaggccaagcttgatcctttaggtctagagaagagagagattccagaggatctcacgccaggtctttatgggtttactgaggctgatcttgatcgggaattctttctgggtgtatggaggatgtcgggttttctctctgagaaccgcccggttcaaacactgaggtcgatactgtcgaggcttgagcaagcttactgtgggactatagggtatgagtacatgcacattgctgatagggataaatgtaactggttgagagacaagatcgagaccccaactcctcgacagtacaatagtgagcgtcggatggttatttatgataggcttacctggagcacacagtttgagaatttcttggctactaagtggaccacggctaaaaggtttggactggaaggtgctgaatctttgattcctggcatgaaggagatgttcgataggtctgcagatctcggggtagagaacatagttatcggtatgccccataggggtcgacttaatgttttgggtaatgttgttagaaaacctctacgccaaatattcagcgagtttagcggtggtactaggccagtagatgaagttgggctttacaccggaacaggtgatgtgaaataccacttgggtacatcttatgatcgtccaactagaggaggcaaacatctccacttgtctttggtagcaaatcccagtcacttggaagcagtagatcctgttgtgataggtaaaaccagagcgaaacaatattacacgaaagacgagaacagaacaaagaacatgggtattttgatccatggggatggtagctttgccggacaaggagtggtgtatgaaactctccatcttagtgcacttcctaactactgtaccggtggaacagtgcacattgtggtgaataatcaagtggctttcacaaccgatcccagggaaggaaggtcttcacagtattgcactgatgttgcaaaggctttgagcgccccaattttccatgtcaatgcagatgacattgaagcagtagtgcatgcttgtgagcttgctgctgagtggcgccagacgttccattctgatgttgttgttgatttagtatgctaccgtcgctttgggcataacgagatagacgaaccgtcattcacacaaccaaaaatgtacaaggtgatacgcagtcatccctcgtcacttcaaatctaccaggagaagctcttgcaatctggacaggtaacccaagaagatattgataagattcaaaagaaagtaagctctatcctcaatgaagaatatgaggcaagtaaagattatattccacaaaaacgtgactggctggcaagtcactggactggattcaagtctccggagcagatttctaggattcgaaacaccggagtgaagccagagattttgaagaatgtgggaaaggcaatctcaaccttccctgagaactttaagccacacagaggagttaaaagagtttatgaacaacgtgctcaaatgattgaatcgggagaaggcattgactggggacttggagaagcacttgcttttgctacactggttgtggaagggaaccatgttcggctaagtggtcaagatgttgaaagaggaactttcagtcatagacactcagtgcttcatgatcaagaaaccggggaggaatattgtcccctcgatcacctaatcaaaaaccaagaccctgaaatgttcactgtcagcaacagctccctttcagaatttggtgttctcggtttcgaactgggttattcgatggaaaatcccaattctctggtgatatgggaagctcagtttggagactttgctaatggcgcacaagttatgtttgatcagttcataagcagtggggaagccaaatggctccgtcaaactggtctagtagttttacttcctcatggatatgatggtcagggtcctgaacattccagtggaagattggaacgtttccttcagatgagtgatgacaatccttacgttatccctgagatggacccaactcttcgaaagcagattcaagaatgtaattggcaagttgttaatgttactacacctgccaactatttccatgttctgcgtcggcagatacacagggactttcgcaagcctcttatagtgatggcccccaaaaacttgcttcgtcacaaacagtgtgtatctaatctctcggaattcgatgatgttaaaggacatcctggatttgacaagcaaggaactcgatttaaacggttgatcaaagatcaaagtggccactctgatcttgaagaaggtatcagacgtctagtcctctgctctgggaaggtctactatgagcttgacgaagagcgaaagaagtctgaaacaaaggatgtagccatttgcagagtagagcagctttgcccatttccatatgatctcatccaaagagaactaaagcgatatccaaatgcagagatcgtgtggtgtcaagaagagccgatgaacatgggaggataccaatacatagccctaaggctttgcaccgcgatgaaagcactgcaaagaggaaacttcaacgacatcaaatacgttggtcgtcttccctcagctgctacagccacaggattttaccagcttcatgttaaggagcagactgatcttgtgaagaaagctcttcaacctgaccccatcacccccgtcatcccttaaaaaaacacagcttgagaggcttgagcctgtataaaaaagacacaacacaaaaataaaagattcatgagagaatctttggttaccaaagagtgtcactggaaaataaacagatgtttgctagacttacaaatttaagtttattcgatttgtttggtttgttataggatttaatcgagataaaaggaaaaaagatttaaaccgtttggtttagtatgataattcattaatttggttcaactaaaaaaaaaaaaaaaaaa

SEQ ID NO: 7 - protein, 1025 aa, Arabidopsis thaliana
MVWFRIGSSVAKLAIRRTLSQSRCGSYATRTRVLPCQTRCFHSTILKSKAESAAPVPRPVPLSKLTDSFLDGTSSVYLEELQRAWEADPNSVDESWDNFFRNFVGQASTSPGISGQTIQESMRLLLLVRAYQVNGHMKAKLDPLGLEKREIPEDLTPGLYGFTEADLDREFFLGVWRMSGFLSENRPVQTLRSILSRLEQAYCGTIGYEYMHIADRDKCNWLRDKIETPTPRQYNSERRMVIYDRLTWSTQFENFLATKWTTAKRFGLEGAESLIPGMKEMFDRSADLGVENIVIGMPHRGRLNVLGNVVRKPLRQIFSEFSGGTRPVDEVGLYTGTGDVKYHLGTSYDRPTRGGKHLHLSLVANPSHLEAVDPVVIGKTRAKQYYTKDENRTKNMGILIHGDGSFAGQGVVYETLHLSALPNYCTGGTVHIVVNNQVAFTTDPREGRSSQYCTDVAKALSAPIFHVNADDIEAVVHACELAAEWRQTFHSDVVVDLVCYRRFGHNEIDEPSFTQPKMYKVIRSHPSSLQIYQEKLLQSGQVTQEDIDKIQKKVSSILNEEYEASKDYIPQKRDWLASHWTGFKSPEQISRIRNTGVKPEILKNVGKAISTFPENFKPHRGVKRVYEQRAQMIESGEGIDWGLGEALAFATLVVEGNHVRLSGQDVERGTFSHRHSVLHDQETGEEYCPLDHLIKNQDPEMFTVSNSSLSEFGVLGFELGYSMENPNSLVIWEAQFGDFANGAQVMFDQFISSGEAKWLRQTGLVVLLPHGYDGQGPEHSSGRLERFLQMSDDNPYVIPEMDPTLRKQIQECNWQVVNVTTPANYFHVLRRQIHRDFRKPLIVMAPKNLLRHKQCVSNLSEFDDVKGHPGFDKQGTRFKRLIKDQSGHSDLEEGIRRLVLCSGKVYYELDEERKKSETKDVAICRVEQLCPFPYDLIQRELKRYPNAEIVWCQEEPMNMGGYQYIALRLCTAMKALQRGNFNDIKYVGRLPSAATATGFYQLHVKEQTDLVKKALQPDPITPVIP

SEQ ID NO: 8 - DNA, 20 bp, Artificial Sequence
  Synthetic DNA
atctcattcagcgtgagctc

SEQ ID NO: 9 - DNA, 29 bp, Artificial Sequence
  Synthetic DNA
cgggtcgacagcaataacaaaactgtata

SEQ ID NO: 10 - DNA, 25 bp, Artificial Sequence
  Synthetic DNA
cgggtacccaagtgtagaacgacga

SEQ ID NO: 11 - DNA, 28 bp, Artificial Sequence
  Synthetic DNA
cgtctagatggtcggttctcagacatga

(19) United States
(12) Patent Application Publication
(10) Pub. No.: US 2002/0100074 A1
(43) Pub. Date: Jul. 25, 2002

(54) Method for producing transformed plant having increased glutamic acid content

(75) Inventors: Yuji Joe, Kawasaki-Shi (JP); Tetsuya Miwa, Kawasaki-Shi (JP); Takao Kida, Kawasaki-Shi (JP); Daisuke Igarashi, Kawasaki-Shi (JP); Chieko Ohsumi, Kawasaki-Shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: AJINOMOTO CO., INC., Chuo-Ku (JP)

(21) Appl. No.: 09/897,056

(22) Filed: Jul. 3, 2001

(30) Foreign Application Priority Data
Jul. 17, 2000 (JP) 2000-215279

(51) Int. Cl. C12N 15/82; A01H 1/00; A01H 5/00

(52) U.S. Cl. 800/278; 800/286; 800/306

(57) ABSTRACT

An object of the present invention is to provide a method for producing a transformed plant whose free glutamic acid content is increased. Moreover, it is a further object of the present invention to provide a transformed plant, whose free glutamic acid content is increased, progeny plants thereof and seeds thereof.

The method of the present invention comprises the steps of transforming a plant with a nucleic acid construct capable of controlling the expression of OGDH gene, and then selecting or identifying the transformed plants based on the presence of the expression of a marker gene present on the nucleic acid construct to thus screen the plant having an increased free glutamic acid content.

### BACKGROUND OF THE INVENTION

The present invention relates to a method for the production of a transformed plant whose free glutamic acid content is increased.

Glutamic acid as an α-amino acid is in general extensively present in proteins and has been known as a component governing the taste and flavor of tomato and components governing the taste and flavor of fermented foods made of, for instance, soy beans. It has been known that glutamic acid is synthesized at the initial stage of the nitrogen assimilation in the higher plant, thereafter glutamine or asparagine originated from glutamic acid are transported to every tissues of the plant body through the sieve tube thereof and then they are used in the synthesis of other amino acids and proteins in the tissues. As has been discussed above, glutamic acid is an amino group donor and is metabolized in a variety of biosynthesis pathways. Accordingly, it is not easy to increase the free glutamic acid content in the plant body and therefore, there have conventionally been known only a small number of examples in which the free glutamic acid content in, for instance, roots of tobacco or corn plants is increased by the incorporation of a gene coding for glutamate dehydrogenase into the same.

Increasing the content of 2-oxoglutaric acid has been considered as a means for increasing the free glutamic acid content in plant's bodies. This is because, the glutamine synthetase-glutamic acid synthase pathway requires 2-oxoglutaric acid as a carbon skeleton. In particular, as an effective means for increasing the free glutamic acid content in microorganisms, there has been proposed a method for inhibiting an enzyme or 2-oxoglutarate dehydrogenase (OGDH), which oxidizes 2-oxoglutaric acid in the TCA cycle to thus produce succinyl CoA(Japanese Un-Examined Patent Publication No. Hei 7-203980). According to this report, in mutants where OGDH activity is defective or reduced, the pathway from 2-oxoglutaric acid to succinyl CoA in the TCA cycle is inhibited, and therefore, the progress of the biosynthesis pathway from 2-oxoglutaric acid to succinyl CoA is improved and the mutant is improved in the ability of producing glutamic acid.

However, it has been known that the production of glutamic acid from 2-oxoglutaric acid in the plant is taken place in the chloroplast and that the glutamic acid-producing site in the plant is considerably different from that observed for the foregoing microorganisms. As the 2-oxoglutaric acid supply pathway in the plant, there has been known a pathway in the cytoplasm wherein 2-oxoglutaric acid is likewise synthesized by the action of isocitrate dehydrogenase (ICDH) from citric acid, through isocitric acid, in addition to the TCA cycle in the mitochondrion or citric acid-isocitric acid-2-oxoglutaric acid pathway in the TCA cycle. It has been reported that the activity of cytoplasm isocitrate dehydrogenase reaches 95% of the total activity of ICDH in tobacco-chlorenchyma and that the citric acid-isocitric acid-2-oxoglutaric acid pathway mainly contributes to the glutamic acid production in the plant's chloroplast. On the other hand, there has been known such a report that, in the tobacco whose ICDH of the cytoplasm is inhibited by an antisense RNA, the concentrations of 2-oxoglutaric acid and free glutamic acid therein do not undergo any change (Kruse, A. et al., Planta, 1998, 205:82-91). However, the pathway from 2-oxoglutaric acid to succinyl CoA, in connection with the biosynthesis pathway of glutamic acid has not yet been regarded as important and thus the OGDH gene involved in this pathway has not positively been genetically engineered. The OGDH gene has not been subjected to any gene engineering as described above, while although the genome structures of at least two genes presumed to be OGDH genes of plants have been known for OGDH E1 subunit, any correct cDNA sequence for the one gene has not yet been known and in any case, there have not yet been investigated functions of the proteins encoded by the genes and roles thereof in the glutamic acid production.

### SUMMARY OF THE INVENTION

It is an object of the present invention to provide a method for producing a transformed plant whose free glutamic acid content is increased. Moreover, it is a further object of the present invention to provide a transformed plant whose free glutamic acid content is increased, progeny plants thereof and seeds thereof.

The inventors of this invention have paid attention to 2-oxoglutaric acid produced in a mitochondrion and the role of 2-oxoglutarate dehydrogenase (OGDH), have found that the free glutamic acid content in plants can be increased by the inhibition of this activity, in particular, the inhibition of the expression of OGDH E1 subunits and thus have completed the present invention.

Accordingly, the present invention relates to a method for producing a transformed plant whose free glutamic acid content is increased as compared with a naturally occurring plant of the same species cultivated under the same conditions, which comprises the step of inhibiting the expression of a gene coding for 2-oxoglutarate dehydrogenase.

More specifically, the present invention relates to a method for producing a transformed plant whose free glutamic acid content is increased as compared with a naturally occurring plant of the same species cultivated under the same conditions, which comprises the steps of transforming a plant with a nucleic acid construct for controlling the expression of OGDH, and then screening the transformed plant based on the expression of a marker gene present on the nucleic acid construct as an indication to thus select or identify the transformed plant having an increased free glutamic acid content.

In particular, the present invention relates to a method characterized in that the foregoing nucleic acid construct is one comprising an antisense RNA against the OGDH E1 subunit gene and a regulator sequence, which can express the antisense RNA.

Moreover, the present invention also relates to progeny plants whose free glutamic acid content is increased and which are originated from the transformed plant whose free glutamic acid content is increased and which is produced by the foregoing method as well as seeds of the progeny plants wherein a nucleic acid construct for controlling the expression of OGDH is operably incorporated into the genome thereof.

### BRIEF DESCRIPTION OF THE DRAWINGS

FIG. 1 shows the structure of a plasmid pSA1AS1.

FIG. 2 shows the structure of a plasmid pBIKm-SA1AS1.

FIG. 3 shows the free amino acids contents in a transformed plant whose ogd1 expression is inhibited.

FIG. 4 shows the overall amino acids contents of a transformed plant (OGD1 No. 3) whose ogd1 expression is inhibited.

FIG. 5 shows the 2-oxoglutalate content of a transformed plant (OGD1 No. 3) whose ogd1 expression is inhibited.

### DESCRIPTION OF THE PREFERRED EMBODIMENTS

An object of the present invention is to increase the free glutamic acid content in plants. To this end, the present invention inhibits the expression of a gene coding for 2-oxoglutarate dehydrogenase (OGDH), which converts 2-oxoglutaric acid into succinyl CoA in the TCA cycle. Such inhibition can be effected by transforming a plant with a nucleic acid construct for inhibiting the expression of OGDH such as that described in this specification. The resulting transformed plant and the progeny plants thereof are screened on the basis of, for instance, the expressed amount of mRNA corresponding to OGDH, the amount of OGDH protein, and the free glutamic acid content in the plant body.

In short, the transformed plant whose free glutamic acid content is increased according to the present invention can be produced by the procedures given below:

a) Incorporating a nucleic acid construct for inhibiting the expression of 2-oxoglutarate dehydrogenase (OGDH) into a plant cell or a plant body and selecting the resulting transformant;

b) Depending on the purposes, regenerating the transformant into a plant body or harvesting seeds to thus obtain transformed plants;

c) Screening the transformed plants obtained in the step b) and further selecting the transformed plant whose free glutamic acid content is increased;

d) If necessary, collecting the progeny plants or seeds of the transformed plants obtained in the step c);

e) Evaluating the progeny plants or seeds obtained in the step d) for the free glutamic acid content to thus obtain progeny plants or seeds of the plants whose free glutamic acid content is increased.

In the present invention, the expression of the OGDH gene is inhibited. The expression of all of the genes coding for every subunits constituting OGDH may be inhibited, but the expression of only one kind of subunit may be inhibited. In general, it is sufficient to inhibit the expression of only one gene coding for one subunit, but the expression of a plurality of subunits may be inhibited depending on the plant species to be engineered and the degree of desired effects. When intensively inhibiting the expression, it is preferred to inhibit the expression of subunits, which are not common to other enzymes or factors playing important roles in other metabolic systems or which interact therewith to only a small extent, among the subunits constituting OGDH. The inhibition of the expression may be carried out at the transcriptional level including the disruption of a gene or at the post-transcriptional level, but it is preferred to control the degree of the inhibition. The inhibition of the expression can likewise be carried out according to the expression of an antisense RNA or so-called gene silencing methods such as the expression of gene-specific double stranded RNA (Chiou-FenCHuang et al., PNAS, 2000, 97:4985-4990) and the co-suppression through intensive expression of the sense sequence (The Plant Journal, 1998, 16:651-659). In any case, the overall length or a part of the OGDH gene can be used. The term “OGDH gene” used in this specification means a generic name of genes coding for any of OGDH subunits unless otherwise specified. Moreover, the term “OGDH mRNA” means a generic name of mRNA's corresponding to each subunit unless otherwise specified.

For instance, the antisense sequence can be expressed by combining an appropriate promoter with the full length or a part of the OGDH gene in the antisense direction or the direction opposed to the original direction of transcription. The double stranded RNA can be formed by conversely arranging the full length or a part of the OGDH genes embracing an appropriate spacer sequence therebetween and connecting them to an appropriate promoter to thus make it express. In this case, the transcribed RNA forms the double stranded RNA in such a manner that the spacer sequence undergoes looping out. The objects of the present invention can be accomplished by incorporating, into a plant cell or a plant body, a nucleic acid construct, which allows such an antisense sequence or the double stranded RNA to express. In case where the co-suppression is employed, it is sufficient to make the full gength or a part of the OGDH gene express under the control of a strong promoter. In the method of the present invention, it is preferred to use the inhibition of expression by the antisense RNA from the viewpoint of, for instance, the simplicity of operations.

In this connection, the term “antisense RNA” used in this specification is used for representing a RNA sequence including a sequence complementary to a mRNA or a part thereof. Therefore, if the term “antisense RNA” is used in connection with a specific gene, the term includes RNA containing only a sequence complementary to the full legth sequence of mRNA naturally transcribed from the gene, RNA including only a sequence complementary to a part of the mRNA and RNA's further including a sequence un-complementary to the mRNA in addition to the foregoing sequences. Moreover, the “antisense RNA” may have a plurality of copies of a sequence complementary to mRNAs.

The nucleic acid construct usable in the present invention can be prepared using a method well-known to those skilled in the art. Regarding molecular biological means including the isolation of the nucleic acid construct and the method for determining the sequence thereof, one can refer to articles such as Sambrook et al., Molecular Cloning-Laboratory manual, 2^{nd} edition, Cold Spring Harbor Laboratory Press. In addition, the production of the nucleic acid construct used in the present invention would often require the gene amplification represented by the PCR method. Such methods are detailed in, for instance, F. M.

Ausubel et al. (eds), Current Protocols in Molecular Biology, John Wiley & Sons, Inc. (1994).

In general, the nucleic acid construct used in the present invention may further contain, in addition to the OGDH gene or a part thereof, an appropriate promoter functional in plant cells such as a nopaline synthase gene or 35S promoter of cauliflower mosaic virus, an appropriate terminator such as the terminator of nopaline synthase gene, other sequences required or effective for the expression, and a marker gene used for screening the desired transformant, for instance, drug-resistant gene such as kanamycin-resistant gene, G418-resistant gene and hygromycin-resistant gene.

The promoter usable in such a construct may be a constitutive promoter or may be organ-specific or growth stage-specific one and it may be selected depending on the host used, the desired amount of the expression and the organ or the growth stage in which the expression is particularly intended. In a preferred embodiment of the present invention, there are used strong promoters, which undergo expression un-specific to organs and growth stages and a CaMV35S promoter is, for instance, used as such a promoter. Examples of organ-specific promoters usable herein are promoters of ribulose bisphosphate carboxylase (RuBisCo) gene and chloroplast a/b bonding protein gene. The most preferred embodiment of the present invention makes use of an OGDH gene sequence connected to a strong constitutive promoter such as a CaMV35S promoter in the antisense direction.

The method for introducing a gene usable in the present invention is not restricted to any particular one and the methods known to those skilled in the art as ones for introducing a gene into plant cells or plant bodies can be selected depending on the host selected. For instance, a gene-introduction method, using Agrobacterium, is employed in an embodiment of the present invention. A binary vector is desirably used in such a transformant line. In case where Agrobacterium is used, the sequence to be introduced is inserted between the left and right T-DNA border sequences. The appropriate design and construction of such a transforming vector based on the T-DNA has been well known to those skilled in the art. Moreover, the conditions for infecting a plant with Agrobacterium containing such a nucleic acid construct have also been well known to those skilled in the art. As to such techniques and conditions, one can refer to Cell Technology, a separate volume entitled “Experimental Protocol for Model Plants: Edition for Rice Plant and Arabis thaliana” (1996).

In the present invention, other gene-introduction methods can likewise be used. Examples of such gene-introduction methods usable herein are DNA-introduction methods, which make use of polyethylene glycols and calcium; methods for transforming protoplasts according to the electroporation; and transduction methods according to the particle gun.

Plant species, which are subjected to the foregoing gene engineering are not restricted to any specific one, but preferably used herein are plant species whose transformation is easy and whose system for regenerating into the plant bodies has been established. Plants suitably used in the present invention more preferably include, in addition to those possessing the foregoing characteristic properties, plant species for which cultivation techniques for mass-production have been established from the viewpoint of the use of glutamic acid produced. Specific examples of plants suitably used for carrying out the present invention include, in addition to all of the plants belonging to the Cruciferae family, tomato, potato, corn, wheat, rice plants and sugar cane. Moreover, organs and cells, which are subjected to the foregoing gene engineering, are not restricted to any specific one and may be selected depending on, for instance, the specific host selected and the specific gene-introduction method selected. Specific examples thereof are explants of organs, pollens, cultured cells, embryos and plant bodies, but the present invention is not restricted to these specific ones.

Then plant cells or the like engineered according to the foregoing procedures are screened for the transformants thereof. This selection can be effected on the basis of the expression of the marker gene present on the nucleic acid construct used in the transformation. If the marker gene is, for instance, a drug resistant gene, the selection can be carried out by cultivating or growing the plant cells or the like genetically engineered on a culture medium containing an antibiotic or a herbicide in an appropriate concentration. Alternatively, if the marker gene is, for instance, β-glucuronidase gene or luciferase gene, desired transformants can be selected by carrying out screening for the activity thereof. If the transformant thus identified is a subject other than plant body such as a protoplast, a callus or an explant, it is regenerated into a plant body. This regeneration can be carried out using a method, known to those skilled in the art, and used for each particular host plant used. The plant body thus obtained may be cultivated according to the conventional method or under the same conditions used for the un-transformed plant body and then subjected to a procedures for identifying the transformed plant containing the nucleic acid construct according to the present invention such as those, which make use of a variety of molecular biological techniques, in addition to the aforementioned selection on the basis of the marker gene. Such techniques usable in the present invention include, for instance, Southern hybridization or PCR techniques for the detection of the presence of a recombinant DNA insert and the structure thereof and Northern blotting and RT-PCR techniques for the detection or determination of an RNA transcript originated from the introduced nucleic acid construct.

Then the resulting transformed plants are evaluated for the amount of OGDH or each OGDH subunit protein or the amount of OGDH mRNA. For instance, the amount of a protein can be evaluated by a method such as Western blotting method and that of the mRNA can be evaluated by a method such as Northern blotting or quantitative RT-PCR method. Moreover, the OGDH activity can be determined by, for instance, the method of Miller et al. (Miller et al., Biochem. J., 1999, 343:327-334). All of these methods have been well known to those skilled in the art and kits for easily carrying out these methods can likewise be commercially available.

The transformed plant whose reduction in the amount of the OGDH protein, that of the OGDH subunit protein, the OGDH activity or the amount of the OGDH mRNA has thus been confirmed is further inspected for the free glutamic acid content. The free glutamic acid content can be examined by, for instance, breaking the transformed plant or a part thereof into pieces, extracting the plant pieces to give an extract and then treating the extract with an amino acid analyzer. Once a transformed plant whose free glutamic acid content is increased is identified, the transformed plant is further inspected for whether the characters thereof are genetically stably maintained therein or not. To this end, it is sufficient to cultivate the plant body under the usual conditions, to harvest the seeds thereof and to analyze the characters and separation thereof in the progeny thereof. The presence of the introduced nucleic acid construct, the position thereof and the expression thereof can be analyzed according to the same procedures used for analyzing the primary transformant.

In the transformed plant whose free glutamic acid content is increased, the sequences originated from the nucleic acid construct introduced and incorporated into the genome may be in the form of either heterozygote or homozygote. Eithere heterozygote or homozygote can be obtained by crossing or mating, depending on the needs. The sequences originated from the nucleic acid construct incorporated into the genome would be separated according to the Mendelian inheritance rule in the progeny. For this reason, it is preferred to use the homozygous plant in order to obtain progeny plants and seeds thereof from the viewpoint of the stability of the characters. The transformed plants thus obtained can be cultivated under the same cultivation conditions used for the naturally occurring plant of the same species and glutamic acid can be extracted from the whole plants or each organ using the usual extraction conditions.

### EXAMPLES

### Example 1

Preparation of OGDH E1 Subunit cDNA Originated from Arabidopsis thaliana

(1) Preparation of DNA, RNA

The seedlings of Arabidopsis thaliana (Columbia; Col-0) were crushed into pieces together with glass beads (5 g/10 g tissues) in an extraction buffer (7.5 ml/10 g tissues; containing 0.2 M Tris HCI, pH 8.0, 0.1 M EDTA, 1% Na N-laurylsarkosyl, 2 mg/ml proteinase K), followed by subjecting to the EtOH precipitation, separating DNA by a CsCI density gradient centrifugation and recovering the same. Separately, the whole RNA was likewise prepared from the seedlings of Arabidopsis thaliana using a reagent ISOGEN for the preparation of RNA available from Nippon Gene Co., Ltd. according to the attached protocol. Then PolyA RNA was recovered from the resulting total RNA using Oligotex dT30 Super available from Nippon Synthetic Rubber Co., Ltd.

(2) Preparation of Library

When searching for sequences homologous with SUCA amino acid sequence of Escherichia coli (E. coli ) using the published EST data of Arabidopsis thaliana, three sequences were obtained. These three sequences corresponded to 444-(T76109), 561-(H37127) and 682-(H36333) of the E. coli SUCA amino acid sequence. All of these EST sequences were on the order of about 300 b in length and these three sequences did not overlap one another.

Then to obtain a probe for an N-terminal proximal region, 5′-RACE was carried out using polyA RNA prepared from the flower buds of Arabidopsis thaliana as a starting material and 5′ RACE System for Rapid Amplification of cDNA Ends, Ver. 2.0 available from GIBCO BRL Company, according to the attached protocol. In this respect, there were used 5′-GAAGGACAGAATGACGATGA-3′ (SEQ ID: 1) corresponding to the sequence derived from the 196 bases of EST H37127 as GSP1 (Gene Specific Primer), 5′-GTGACGAGGGATGACTGCGT-3′ (SEQ ID: 2) corresponding to the sequence derived from the 110 bases of EST T76109 as GSP2 and 5′-TCGTCTATCTCGTTATGCCC-3′ (SEQ ID:3) corresponding to the sequence derived from the 54 bases of EST T76109 as nested GSP. As a result, a variety of fragments were obtained and the longest one was found to be 1.3 kb in length. The terminal of the fragment of 1.3 kb was blunted before inserting the fragment into the Smal site of a plasmid pBluescript SK+. The resulting plasmid was digested with Sacl and BamHl to form a deletion plasmid and then the base sequence thereof was determined. The resulting fragment was found to have a sequence homologous with E. coli SucA.

(3) Isolation of OGDH Gene of Arabidopsis thaliana

A λ-phage cDNA library was prepared using polyA RNA prepared from the flower buds of Arabidopsis thaliana as a starting material and SUPERSCRIPT Lambda System for cDNA Synthesis and Lambda Cloning Kit available from GIBCO BRL Company according to the attached protocol. According to this kit, each cDNA fragment was inserted into the Sall-Notl site of pZL1 contained in a vector λZ_{lP}Lox. Thus, a library was obtained, which comprised about 200,000 independent clones having a variety of lengths whose median was positioned at about 2 kb. This library was amplified and used in the following experiments.

A probe was prepared using Prime lt-II, [α-³²P]dCTP available from Stratagene Company and a Hincll-Xbal digested fragment (a fragment of about 500 bp) of the deletion plasmid prepared for sequencing the 5′ RACE product obtained above, as a template. After carrying out plaque hybridization at 60° C. according to the method of Church et al. (Church G. M. et al., P.N.A.S. USA, 1984, 74:5350), the hybridization product was washed with 0.2× SSC 0.1% SDS at 60° C. (Hybridization was likewise carried out in the Northern and Southern analysis under the similar conditions, unless otherwise specified).

As a result, two clones were obtained, which comprised cDNA's having different sequences and a length of about 3.5 kb. The sequences of cDNA's included in these clones were determined and it was found that both of them comprised sequences homologous with E. coli SucA. In addition, putative amino acid sequences were compared with OGDH E1 subunits derived from organisms of other species and the upstream sequences were analyzed. As a result, it was found that a stop codon is present in the same upstream reading frame and that it has such a typical sequence that in the proximity to the putative initiation codon, +4-position is G and −3-position is a purine. Accordingly, it was recognized that the cDNA contained in these clones encompass the full length of the translational region of the OGDH gene. From these results, it was suggested that the genes contained in these two clones are the two genes encoding for OGDH E1 subunits of Arabidopsis thaliana. The putative amino acid sequences had a homology with OGDH's from other species, of about 40%. These two genes will hereafter be referred to as “OGD1 gene” or “ogd1” and “OGD2 gene” or “ogd2”, respectively. The plasmid in which each cDNA was inserted into the Sall-Notl region was spliced from each clone corresponding to odg1 or odg2 using a deletion/recyclized system (the aforementioned kit available from GIBCO BR Company), which made use of the Cre-loxP recombination to thus give a plasmid pAtOGD1 or pAtOGD2. The base sequences of ogd1 and ogd2 cDNA's included in these plasmids are shown in SEQ ID: 4 and SEQ ID:6, while the amino acid sequences presumed on the basis of these cDNA's are likewise shown in SEQ ID: 5 and SEQ ID:7, respectively.

### Example 2

Inhibition of Expression of OGDH Gene in Arabidopsis thaliana

(1) Construction of Plasmid for Antisense Sequence Expression

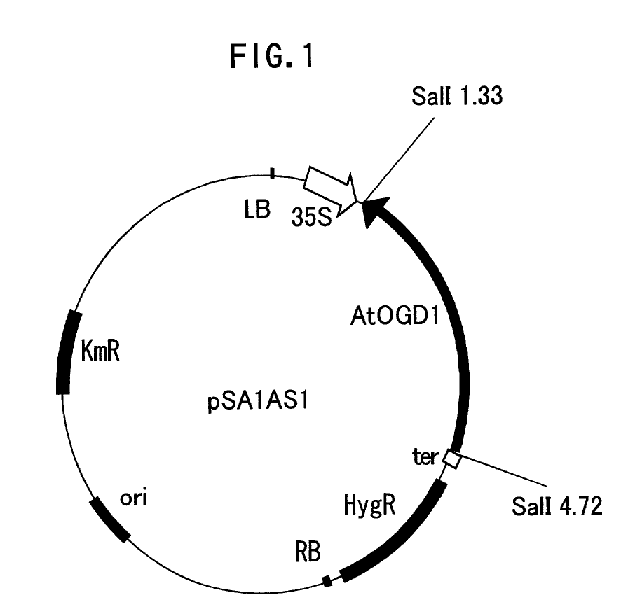

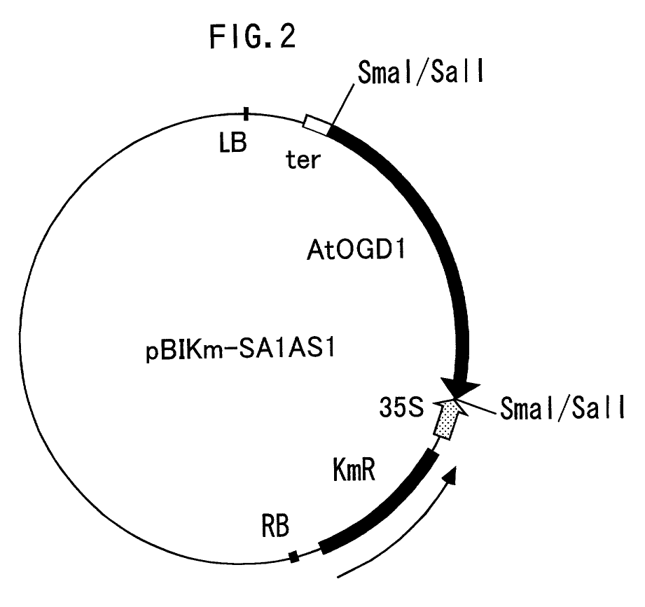

In this Example, pBIKm and pBINHYGTOR (Hofgen et al., P.N.A.S., 91:1726-1730) were used as binary vectors carrying a CaMV35S promoter. In this respect, pBIKm is one in which the uidA site of pBI121 is substituted for the Smal-Sacl site of pUC18 polylinker. The plasmid for expressing an antisense RNA corresponding to the whole length of ogd1 cDNA was constructed according to the following procedures. An EcoRV-Sall digestion fragment of the PCR products corresponding to the C-terminal portion of OGD1 obtained using pAtOGD1 discribed in Example 1 as a template was introduced into the Sall site of pBluescriptSK+ together with the Sall-EcoRV digested fragment (including the region on the N-terminal region of OGD1) of the plasmid pAtODG1, while using a primer: 5′-CGGGTCGACAGCAATAACAAAACTGTATA-3′ (SEQ ID: 9; the portion of the ogd1 sequence corresponds to the 3379^{th} to 3360^{th} nucleotides in SEQ ID: 4) to which a primer: 5′-ATCTCATTCAGCGTGAGCTC-3′ (SEQ ID: 8; the portion corresponding to the 2900^{th} to 2919^{th} nucleotides in SEQ ID: 4) and the Sall site were added and the Sall fragment of the resulting plasmid was inserted into the plasmid pBINHYGTOR to give a plasmid pSA1AS1 (FIG. 1). After blunting the fragment, it was inserted into the Smal site of pBIKm to give a plasmid pBIKm-Sa1AS1 (FIG. 2). These plasmids comprise the complete fragment corresponding to 1^{st} to 3379th nulcleotides of SEQ ID: 4 and have a fragment in which the Sall linker is added to the 5′- and 3′-terminals.

After cloning all of the foregoing PCR products, the nucleotide sequences thereof were confirmed prior to the final plasmid construction.

(2) Introduction of Antisense Plasmid-Expressing Plasmid into Plants

Two kinds of the foregoing plasmids were introduced into Agrobacterium C58C1 Rif according to the triparental mating technique using E. coli and helper E. coli strain HB101/pRK2013 carrying the plasmids pSA1AS1 and pBIKm-SA1AS1. A plant was then infected by the infiltration under reduced pressure technique using the resulting Agrobacterium C58C1Rif carrying the plasmid pSA1AS1 or pBIKm-Sa1AS1 according to the procedures disclosed in Cell Technology, a separate volume entitled “Experimental Protocol for Model Plants: Edition for Rice Plant and Arabis thaliana”, published by SHUJUNSHA Publishing Press (1996). In case of the infiltration under reduced pressure technique, the seeds harvested from the infiltrated plant correspond to the T1 plants and therefore, the primary screening thereof was carried out by seeding these sterilized seeds on a GM agar medium (1×MS, 1×B5 vitamin, 10 g/l sucrose, 0.5 g/l MES-KOH (pH 7.5), 0.8% agar) containing 20 mg/l of hygromycin B (or 50 mg/l of Kanamycin sulfate). T2 Seeds were harvested from the-plants grown on the culture medium. The T1 plant is a heterozygote and therefore, the T2 plants grown from the T2 seeds should undergo separation in accordance with the Mendel's law. In fact, this is also true in the experiments in this Example. Thus, a part of the seeds originated from the T2 plants were seeded to examine the resistance to hygromycin of the T3 plants and the line in which all of the T3 plants were hygromycin-resistant was maintained as a homozygous line with respect to the introduced gene.

Then genome Southern analysis was carried out using 8 lines (6 homogeneous lines and 2 heterogeneous lines) of the T3 plants derived from each T1line of plants, which were expected to have a sequence constructed so as to express an RNA having the ogd1 antisense sequence. The probe used herein was PCR products obtained using primers 5′-CGGGTACCCAAGTGTAGAACGACGATTG-3′ (SEQ ID: 10) and 5′-CGTCTAGATGGTCGGTTCTCAGACATGA-3′ (SEQ ID: 11) and using pOGD1 as a template. After digesting 200 ng of genome DNA with Hind III, Southern blotting analysis was performed using the foregoing fragment as a probe. In this respect, the hybridization was effected according to the method of Church et al. (Church, G. M. et al., P.N.A.S. USA, 1984, 74:5350). As a result, it was confirmed that the sequence used for expressing the antisense sequence was incorporated into the genome.

(3) Evaluation of Amounts of mRNA and Protein of OGDH E1 Subunit

The amount of mRNA present in ogd1 as one of genes coding for OGDH E1 subunits observed for the transformed plant was compared with that observed for non-transformed plant. Then Northern blotting analysis was effected using 10 μg of the whole RNA prepared from the rosette leaves of Arabidopsis thaliana. The antisense probe or the probe for detecting ogd1 mRNA was prepared by digesting a plasmid constructed by introducing the same PCR product used in the Southern blotting analysis in Section (2) into the plasmid pBluescriptSK+ and using T3 polymerase with In Vitro Transcription Kit available from Stratagene Company. In this connection, the hybridization was carried out using the buffer having a composition specified in the explanatory note attached to In Vitro Transcription Kit available from Stratagene Company under the conditions described therein.

As a result, in 3 lines out of 6 lines, which were proved to be homozygotes based on the separation pattern of the hygromycin resistant plant body, there was observed a considerable signal reduction in the band of about 3.5 kb and this indicates that the amount of OGD1 mRNA is substantially reduced.

Then these three lines in which it was proved, in the Northern blotting analysis, that OGD1 mRNA content was reduced were inspected for the amount of ogd1 translated products or the amount of OGD1 proteins.

For the purpose of effecting Western blotting analysis, a rabbit anti-serum against a partial peptide of OGD1 expressed in E. coli host was prepared. We entrusted Takara Shuzo Co., Ltd. with the preparation thereof. In this respect, an antigen used herein was a peptide obtained by adding Met-Ser-Phe- to the N-terminal of the OGD1 partial peptide corresponding to the 38^{th} to 512^{th} residues of the amino acid sequence described in SEQ ID:5. The fragment: Met-Ser-Phe- is not a sequence derived from OGD1, but a sequence artificially added thereto during the construction of a partial peptide-expressing construct in E. coli.

The rosette leaf extracts from the foregoing 6 lines of Arabidopsis thaliana were subjected to Western blotting analysis using the resulting anti-serum. The proteins were transferred to a PVDF membrane by carrying out SDS-polyacrylamide gel electrophoresis according to the method of Laemmli et al. The proteins were transferred using the buffer of Towbin (Towbin, H. et al., P.N.A.S., 1979, 76:4350) in a Sartoblotil device available from Sartorius Company. The proteins on the membrane were detected using the foregoing anti-serum and Immune Blotting Kit available from Bio-Rad Company according to the method recommended by the manufacturer.

As a result, there was observed a decrease of the OGD1 content in the lines whose mRNA content was reduced in the Northern analysis, but there was not observed any reduction of the OGD1 content in the lines, which did not show any reduction of the mRNA content. More specifically, it was found that the reduction of the mRNA content accompanied by the antisense RNA expression (as determined in the Northern blotting analysis) was strongly correlated with the reduction of the OGD1 protein content.

### Example 3

Evaluation of Free Glutamic Acid Content, Overall Amino Acid Content and 2-Oxoglutaric Acid Content in Plant Whose Expression of OGDH E1 Subunit is inhibited

The transformed plant lines, which showed a decrease of the OGD1 mRNA content were grown over 4 weeks in an air-conditioned greenhouse (photoperiod: 14 hours; temperature: 23° C.). The leaves of each transformed plant line were milled using a mortar and a pestle in liquid nitrogen, followed by extraction thereof with 80% ethanol at 70° C., concentration of the extract from which proteins were removed through ultrafiltration, dilution of the concentrate with a 0.01 N hydrochloric acid solution and determination of the amount of free amino acids-using an amino acid analyzer L8800 available from Hitachi, Ltd.

The extraction of keto acid and the derivatization with 2,4-dinitrophenyl hydrazine were carried out according to the procedures disclosed in the literature (Slater et al., Nature Biotech., 17:1011-1016) and then free amino acids and 2-oxoglutaric acid were separated from one another and quantitatively analyzed using ODS-80TM available from Tosoh Corporation and 60% ethanol and 1.5% acetic acid solutions as solvents.

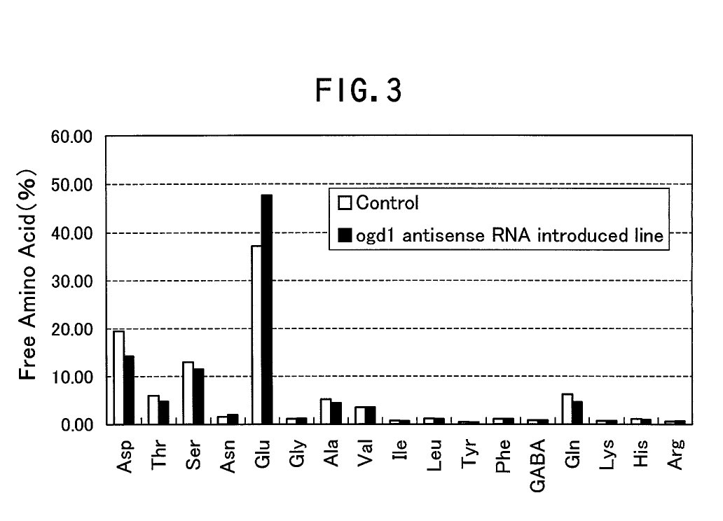

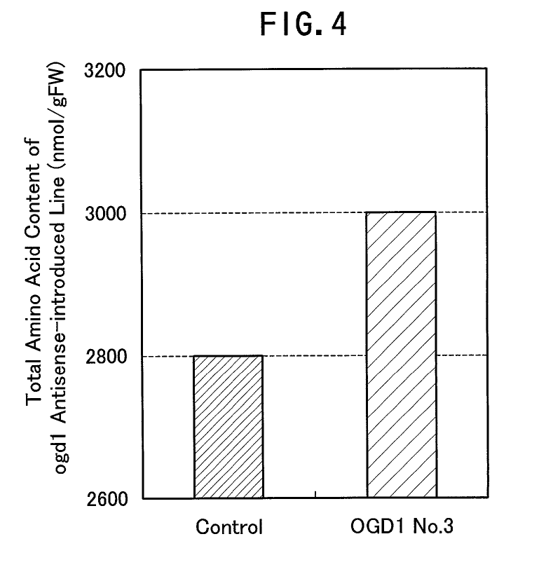

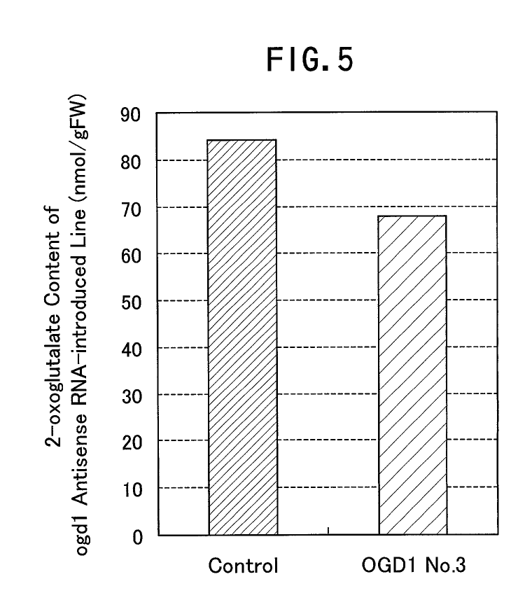

Typical results of these determinations are summarized in the following Tables 1 to 3 and plotted on FIGS. 3 to 5.

TABLE 1
Free Amino Acid Composition (%) of Transformed Strain

| | | ogd1 Antisense RNA-Introduced Plant |
|---|---|---|
| | Control Plant | Line (OGD1 No.3) |
| Asp | 19.55 | 14.21 |
| Thr | 6.08 | 4.80 |
| Ser | 13.07 | 11.57 |
| Asn | 1.44 | 1.91 |
| Glu | 37.16 | 47.68 |
| Gly | 1.07 | 1.13 |
| Ala | 5.31 | 4.48 |
| Val | 3.51 | 3.56 |
| Ile | 0.72 | 0.63 |
| Leu | 1.15 | 1.03 |
| Tyr | 0.43 | 0.38 |
| Phe | 1.07 | 1.01 |
| GABA | 0.80 | 0.80 |
| Gln | 6.33 | 4.63 |
| Lys | 0.69 | 0.65 |
| His | 1.07 | 0.91 |
| Arg | 0.56 | 0.63 |

TABLE 2
Overall Amino Acid Content of Transformed Strain (nmol/gFW)

| | ogd1 Antisense RNA-Introduced Plant |
|---|---|
| Control Plant | Line (OGD1 No.3) |
| 2800 | 3000 |

TABLE 3
2-Oxoglutaric Acid Content of Transformed Strain (nmol/gFW)

| | ogd1 Antisense RNA-Introduced Plant |
|---|---|
| Control Plant | Line (OGD1 No.3) |
| 84 | 68 |

The foregoing results indicate that the free glutamic acid content of a plant body is significantly increased in the line whose expression of ogd1 is inhibited by the action of ogd1 antisense RNA.

The present invention permits the production of a transformed plant whose free glutamic acid content is increased. The present invention can provide a transformed plant whose free glutamic acid content is increased by at least 20%.

It is also understood that the examples and embodiments described herein are only for illustrative purpose, and that various modifications will be suggested to those skilled in the art without departing from the spirit and the scope of the invention as hereinafter claimed.

What is claimed is:

1. A method for producing a transformed plant whose free glutamic acid content is increased as compared with a naturally occurring plant of the same kind cultivated under the same conditions, comprising the steps of transforming a plant with a nucleic acid construct for controlling the expression of 2-oxoglutarate dehydrogenase (OGDH), and then screening the transformed plant based on the phenotype conferred to the plant by a marker gene present on the nucleic acid construct to thus select the transformed plant having an increased free glutamic acid content.

2. The method of claim 1, wherein the nucleic acid construct for controlling the expression of 2-oxoglutarate dehydrogenase (OGDH) is a nucleic acid construct comprising an antisense RNA against the full length of the cDNA sequence of a gene encoding OGDH or a partial sequence thereof and a regulator sequence, which can express, in plant's cells, the antisense RNA sequence.

3. The method of claim 1, wherein the nucleic acid construct controls the expression of OGDH E1 subunit.

4. The method of claim 1, wherein the nucleic acid construct contains the sequence of nucleotide nos.1-3379 of SEQ ID:4 or its complementary sequence.

5. The method of claim 2, wherein the antisense RNA is an antisense RNA against a coding region in the cDNA of the gene coding for OGDH.

6. The method of claim 2, wherein the antisense RNA is an antisense RNA against OGDH E1 subunit mRNA.

7. A plant produced by the method of claim 1.

8. A plant produced by the method of claim 2.

9. A plant produced by the method of claim 3.

10. A plant produced by the method of claim 4.

11. A progeny plant of the transformed plant of claim 7 and whose free glutamic acid content is improved as compared with that observed for a naturally occurring plant of the same kind cultivated under the same conditions.

12. A progeny plant of the transformed plant of claim 8 and whose free glutamic acid content is improved as compared with that observed for a naturally occurring plant of the same kind cultivated under the same conditions.

13. The plant of claim 7, wherein the plant is one belonging to Cruciferae.

14. The plant of claim 8, wherein the plant is one belonging to Cruciferae.

15. A seed of the plant of claim 1, wherein a nucleic acid construct for controlling the expression of 2-oxoglutarate dehydrogenase (OGDH) is operably incorporated into the genome thereof.

16. A seed of the plant of claim 2, wherein a nucleic acid construct for controlling the expression of 2-oxoglutarate dehydrogenase (OGDH) is operably incorporated into the genome thereof.
